(19) United States
(12) Patent Application Publication
(10) Pub. No.: US 2010/0121299 A1
(43) Pub. Date: May 13, 2010

(54) WRAPPING PACKAGE

(76) Inventor: Karen Cooper, Cortland Manor, NY (US)

Correspondence Address: JAY M. SCHLOFF, 6960 Orchard Lake Road, Suite 315, West Bloomfield, MI, 48322, US

(21) Appl. No.: 12/511,293

(22) Filed: Jul. 29, 2009

Related U.S. Application Data
(60) Provisional application No. 61/112,467, filed on Nov. 7, 2008.

(51) Int. Cl. A61F 13/551; A61F 13/20

(52) U.S. Cl. 604/385.02; 604/385.13

(57) ABSTRACT

A wrapping package for storing one or more unused articles such as a new tampon, and one or more used articles such as a used tampon, includes a base member, a flap member, and a covering member. The base member includes a first section, a second section, and a middle section configured in between the first section and the second section. An end portion of the flap member is capable of being resealably coupled with the first section for configuring a first enclosure, which is capable of storing the one or more unused articles. The covering member is coupled with the second section of the base member for configuring a second enclosure, which is capable of receiving the one or more used articles. Further disclosed is a method of accessing the one or more unused articles and disposing the one or more used articles.

### CROSS-REFERENCE TO RELATED APPLICATIONS

The present disclosure claims priority under 35 United States Code, Section 119 from the U.S. Provisional Patent Application No. 61/112,467 filed on Nov. 7, 2008, the disclosure of which is incorporated herein by reference.

### FIELD OF THE DISCLOSURE

The present disclosure generally relates to wrapping packages, and, more particularly, to a wrapping package capable of storing one or more unused articles such as a new tampon; and one or more used articles such as a used tampon, separately.

### BACKGROUND OF THE DISCLOSURE

Various feminine care articles such as a tampon, a sanitary napkin, and the like, are known to be used for absorbing body fluids. Specifically, a tampon is inserted into vagina of a woman for absorption of the body fluids discharged during her menstrual cycle. Insertion of a tampon into vagina of a woman is commonly achieved using a tampon applicator that comes initially assembled with the tampon. The tampon applicator, which is often made of plastic or cardboard, is disposable. Thus, the tampon applicator may be discarded in the wrapping in which the new tampon had been enclosed prior to use, for example, after being used for inserting the tampon into a woman's vagina.

For maintaining hygiene, regular replacement and disposal of used tampons is required. Also, proper disposal of the used tampons is necessary for sanitary purposes. It has been observed that generally women flush down the used tampons in a toilet. However, flushing down the used tampons may cause clogging in a draining system of the toilet.

Alternately, used tampons may be disposed into a garbage bin. More specifically, women may wrap the used tampons in multiple layers of toilet paper prior to disposing the used tampons into the garbage bin. However, the disposal of the wrapped tampon in the garbage bin is undesirable as body fluids retained in the used tampons may leak through the multiple layers of the toilet paper, thereby causing unhygienic conditions. Further, the leakage of the body fluids may result in spreading of an unpleasant odor in the environment. Moreover, used tampons, when wrapped improperly may also be seen by other individuals, thereby causing embarrassment for the disposer.

To avoid such an embarrassing situation, women may prefer to carry used tampons in a concealed and discrete fashion, until they can be properly disposed. Thus, to hide and dispose the used tampons, and to avoid unsanitary situations caused by the tampons leaking before they can be disposed, various conventional packages such as tampon wrappers are utilized. However, conventional tampon wrappers are unable to properly enclose and conceal the used tampons due to the soiled conditions thereof caused by the body fluids. Moreover, a woman needs to separately carry a tampon pouch or disposal bag for disposing a used tampon, which may cause inconvenience.

In addition, tampon wrappers have been developed in the form of a single unit for storing unused tampons. Specifically, an individual wrapper may initially contain a tampon assembly having a new tampon that may be withdrawn for use. However, such an individual wrapper is not sufficient for disposing of a used tampon due to the fluids contained and expanded size; rather, only the used tampon applicator from the new tampon (hereinafter referred to as “used tampon applicator”) once removed from the tampon assembly can be received in the individual wrapper. More specifically, a woman needs to take out the tampon assembly from the individual wrapper to use the applicator for inserting the new tampon into her vagina. However, before inserting the new tampon into her vagina, the woman is required to place the new tampon assembly aside, while disposing of the used tampon Keeping the new tampon assembly aside anywhere other than within an enclosed package prior to a use thereof may prove unhygienic for the woman, as the new tampon may become infected or unsanitary when in the open. Further, the individual wrapper is incapable of completely concealing the used tampon and the used tampon applicator wrapped therein.

Accordingly, there exists a need for a single unit wrapping package capable of separately storing one or more unused articles such as a new tampon and a new tampon assembly including a new tampon and a tampon applicator; and one or more used articles such as a used tampon for disposal thereof, in an effective sanitary and convenient manner. There also exists a need for a wrapper package that allows the user to contain and seal a used tampon, while keeping a new tampon and applicator sanitary until the user is ready to use the new tampon and applicator. Further, there exists a need for a wrapping package that is capable of preventing leakage of fluids, such as body fluids, retained in a used article concealed within the wrapping package.

### SUMMARY OF THE DISCLOSURE

In view of the foregoing disadvantages inherent in the prior art, the general purpose of the present disclosure is to provide a wrapping package for storing one or more unused articles such as a new tampon; and one or more used articles such as a used tampon, separately, configured to include all the advantages of the prior art, and to overcome the drawbacks inherent therein.

Therefore, an object of the present disclosure is to provide a wrapping package that is capable of storing one or more unused articles such as a new tampon, and a new tampon assembly including a new tampon and a tampon applicator; and one or more used articles such as a used tampon, separately.

Another object of the present disclosure is to provide a wrapping package that is capable of preventing leakage of fluids, such as body fluids, retained in a used article concealed within the wrapping package.

To achieve the above objects, in an aspect of the present disclosure, a wrapping package is provided. The wrapping package includes a base member that includes a first section having a first plurality of edges. Further, the base member includes a second section having a second plurality of edges. The base member also includes a middle section configured in between the first section and the second section. Further, the wrapping package includes a flap member extending outwardly from the first section of the base member. The flap member includes a first end portion and a second end portion opposite to the first end portion. The first end portion of the flap member is coupled with a first edge of the first plurality of edges of the first section. The second end portion of the flap member is capable of being resealably coupled with a second edge opposite to the first edge of the first plurality of edges for configuring a first enclosure between the flap member and the first section of the base member. The first enclosure is capable of storing one or more unused articles. Furthermore, the wrapping package includes a covering member coupled with the second section of the base member at three edges of the second plurality of edges of the second section for configuring a second enclosure between the covering member and the second section of the base member. The second enclosure is capable of receiving one or more used articles.

In another aspect, the present disclosure provides a method of accessing one or more unused articles and disposing one or more used articles. The method includes opening a wrapping package provided in a closed configuration. The wrapping package includes a base member that includes a first section having a first plurality of edges. Further, the base member includes a second section having a second plurality of edges. The base member also includes a middle section configured in between the first section and the second section. Further, the wrapping package includes a flap member extending outwardly from the first section of the base member. The flap member includes a first end portion and a second end portion opposite to the first end portion. The first end portion of the flap member is coupled with a first edge of the first plurality of edges of the first section. The second end portion is resealably coupled with a second edge opposite to the first edge of the first plurality of edges for configuring a first enclosure between the flap member and the first section of the base member. The first enclosure is capable of storing the one or more unused articles. The flap member includes a first sealing element disposed at the second end portion of the flap member for resealably coupling the second end portion of the flap member with the second edge of the first plurality of edges.

Furthermore, the wrapping package includes a covering member coupled with the second section of the base member at three edges of the second plurality of edges of the second section for configuring a second enclosure between the covering member and the second section of the base member. The second enclosure is capable of receiving the one or more used articles. The covering member includes a second sealing element disposed on a surface of the covering member. The wrapping package assumes a closed configuration when the base member is rolled from the first section towards the second section and the second sealing element resealably couples with a rear surface of the base member. The wrapping package is opened by decoupling the second sealing element and pulling apart the second section and the covering member from the rear surface of the base member for enabling the wrapping package to assume an open configuration.

Further, the method includes inserting a first used article from the one or more used articles into the second enclosure. Furthermore, the method includes decoupling the second end portion of the flap member from the second edge of the first plurality of edges of the first section by pulling apart the second end portion of the flap member away from the second edge of the first plurality of edges. Additionally, the method includes withdrawing the one or more unused articles from within the first enclosure. Moreover, the method includes rolling the base member from the second section towards the first section, such that the first sealing element resealably couples with the rear surface of the base member. In addition, the method includes disposing the wrapping package.

These together with other aspects of the present disclosure, along with the various features of novelty that characterize the present disclosure, are pointed out with particularity in the claims annexed hereto and form a part of this present disclosure. For a better understanding of the present disclosure, its operating advantages, and the specific objects attained by its uses, reference should be made to the accompanying drawings and descriptive matter in which there are illustrated exemplary embodiments of the present disclosure.

### BRIEF DESCRIPTION OF THE DRAWINGS

The advantages and features of the present disclosure will become better understood with reference to the following detailed description and claims taken in conjunction with the accompanying drawings, wherein like elements are identified with like symbols, and in which:

FIG. 1A depicts a front view of a wrapping package in an open configuration, in accordance with an embodiment of the present disclosure;

FIG. 1B depicts a front view of the wrapping package of FIG. 1A in a closed configuration, storing one or more unused articles, in accordance with an embodiment of the present disclosure;

FIG. 2A depicts an exploded view of the wrapping package of FIGS. 1A and 1B, in accordance with an embodiment of the present disclosure;

FIG. 2B depicts a perspective view of the wrapping package of FIG. 2A, storing one or more unused articles, in accordance with an embodiment of the present disclosure;

FIG. 3 depicts a front view of the wrapping package of FIG. 2B, in accordance with an embodiment of the present disclosure;

FIG. 4A depicts a perspective view of the wrapping package of FIG. 3, in a partially closed configuration prior to disposal, in accordance with an embodiment of the present disclosure;

FIG. 4B depicts a perspective view of the wrapping package of FIG. 4A, in a closed configuration prior to disposal, in accordance with an embodiment of the present disclosure; and

FIGS. 5A and 5B depict a flow diagram of a method for accessing one or more unused articles and disposing one or more used articles, in accordance with an embodiment of the present disclosure.

Like reference numerals refer to like parts throughout the description of several views of the drawings.

### DETAILED DESCRIPTION OF THE DISCLOSURE

The exemplary embodiments described herein detail for illustrative purposes are subject to many variations in structure and design. It should be emphasized, however, that the present disclosure is not limited to a particular wrapping package, as shown and described. It is understood that various omissions and substitutions of equivalents are contemplated as circumstances may suggest or render expedient, but these are intended to cover the application or implementation without departing from the spirit or scope of the claims of the present disclosure. Also, it is to be understood that the phraseology and terminology used herein is for the purpose of description and should not be regarded as limiting. The use of terms, “including,” or “comprising,” and variations thereof herein is meant to encompass the items listed thereafter and equivalents thereof as well as additional items.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

Unless limited otherwise, the terms “attached,” “secured,” “coupled,” and variations thereof herein are used broadly and encompass direct and indirect attachments and arrangements. The terms, “top,” “bottom,” “side,” and the like, herein do not denote any order, elevation or importance, but rather are used to distinguish placement of one element over another.

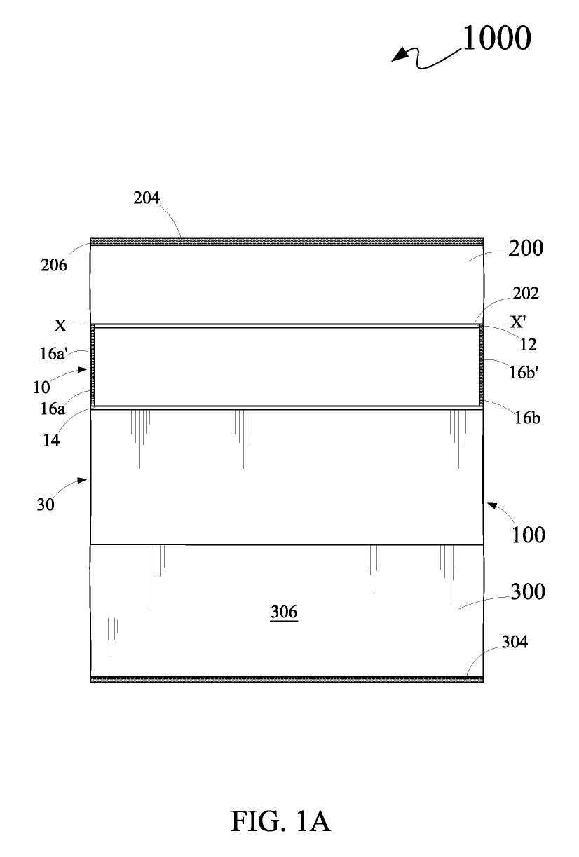

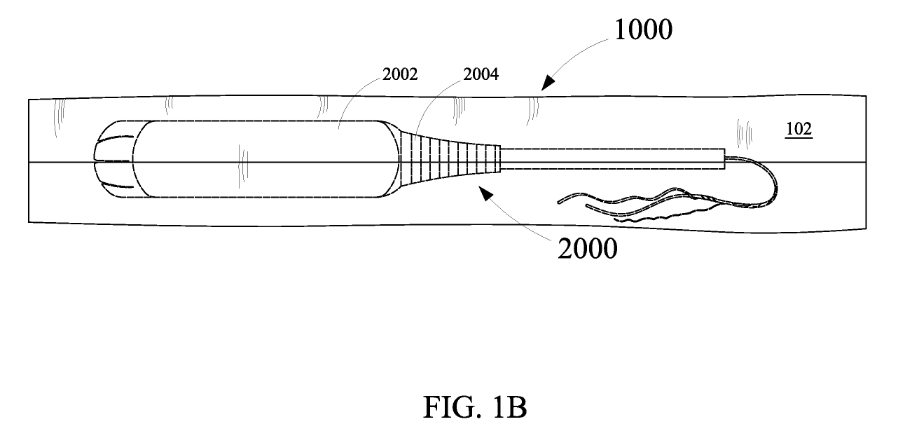

In one aspect, the present disclosure provides a wrapping package for storing one or more unused articles and one or more used articles, separately. The term, “unused articles” relates to female sanitary articles such as a new tampon, a new tampon applicator, and the like. Further, the term, “used articles” relates to used female sanitary articles such as a used tampon, a tampon applicator used for inserting a tampon into vagina of a woman, and the like. Specifically, the wrapping package of the present disclosure allows for separately storing a tampon assembly including a new tampon and a new tampon applicator; and a used tampon and the new tampon applicator after inserting the new tampon into the vagina of a woman. The wrapping package of the present disclosure is explained in detail in conjunction with FIGS. 1A, 1B, 2A, 2B, 3, 4A, and 4B.

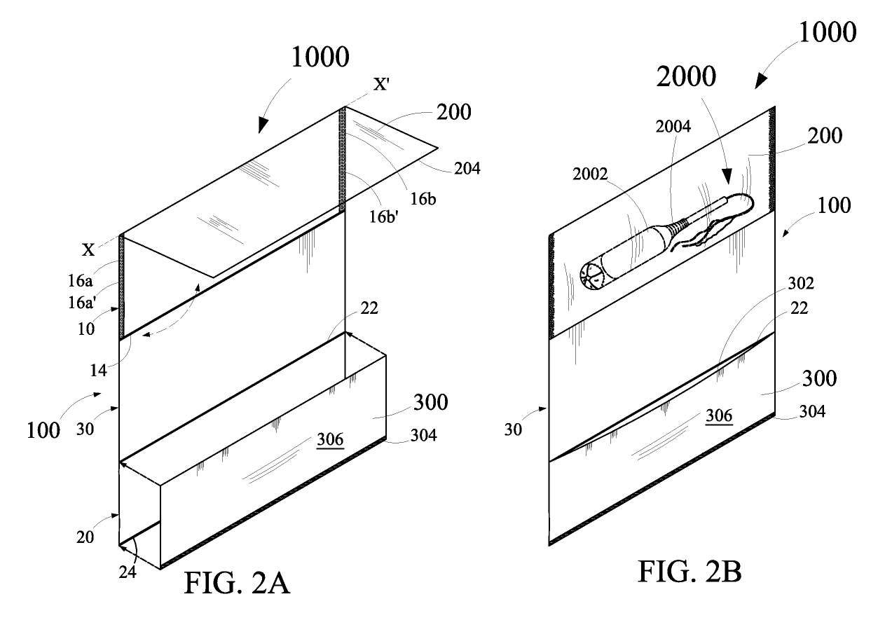

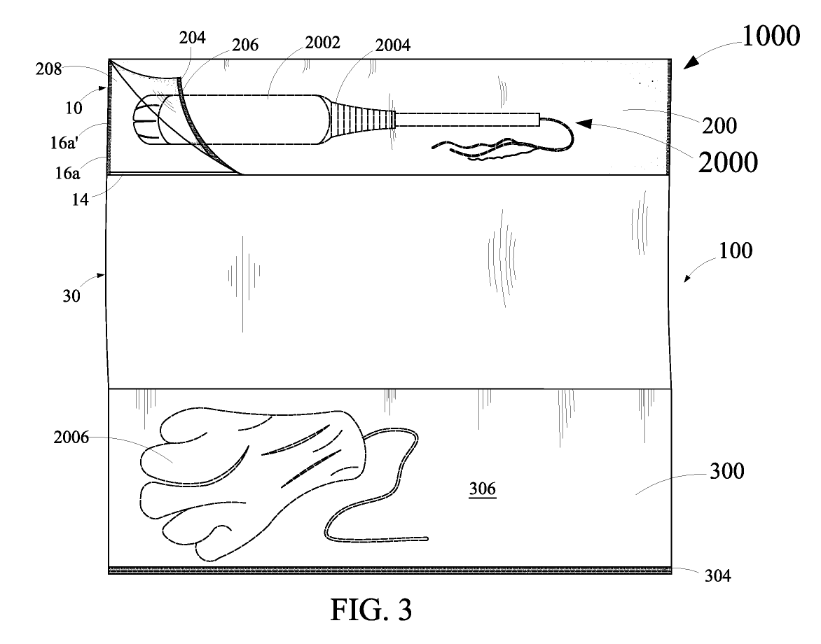

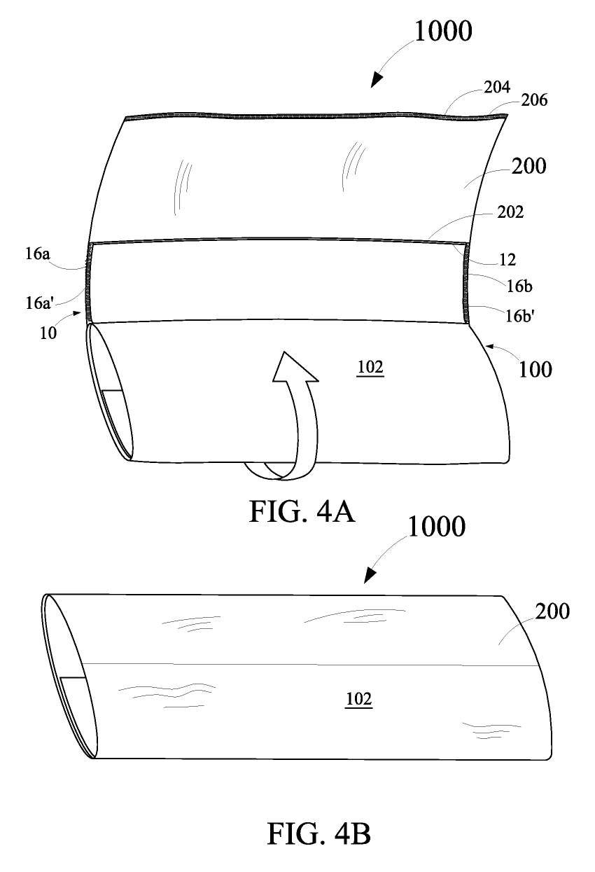

FIG. 1A depicts a front view of a wrapping package 1000 in an open configuration (i.e., an unrolled configuration), in accordance with an embodiment of the present disclosure. Further, FIG. 1A depicts various components of the wrapping package 1000 without depiction of one or more unused articles. FIG. 1B depicts a front view of the wrapping package 1000 in a closed configuration (i.e., a rolled configuration), storing one or more unused articles, in accordance with an embodiment of the present disclosure. FIG. 2A depicts an exploded view of the wrapping package 1000, in accordance with an embodiment of the present disclosure. FIG. 2B depicts a perspective view of the wrapping package 1000, storing the one or more unused articles, in accordance with an embodiment of the present disclosure. FIG. 3 depicts a front view of the wrapping package 1000, in accordance with an embodiment of the present disclosure. FIG. 4A depicts a perspective view of the wrapping package 1000 in a partially closed configuration prior to disposal, in accordance with an embodiment of the present disclosure. FIG. 4B depicts a perspective view of the wrapping package 1000 in a closed configuration prior to disposal, in accordance with an embodiment of the present disclosure.

The wrapping package 1000 includes a base member 100, a flap member 200, and a covering member 300 (as depicted in FIG. 1A). The base member 100 includes a first section 10, a second section 20, and a middle section 30. Further, the first section 10 includes a first plurality of edges such as a first edge 12 (hereinafter referred to as “first top edge 12”), a first pair of side edges 16a and 16b, and a second edge 14 (hereinafter referred to as “first bottom edge 14”) opposite to the first top edge 12. In an embodiment of the present disclosure, the first pair of side edges 16a and 16b may be provided with resealable coupling elements 16a′ and 16b′ (as shown in FIG. 1A) disposed thereon. Examples of the resealable coupling elements 16a′ and 16b′ may include but are not limited to an adhesive strip, a tab of tape, and a layer of glue. Furthermore, the second section 20 includes a second plurality of edges such as a second top edge 22, a second pair of side edges (not numbered), and a second bottom edge 24 (as shown in FIG. 2A). As shown in FIGS. 1A and 2A, the middle section 30 of the base member 100 is configured between the first section 10 and the second section 20. Specifically, the middle section 30 is configured in between the first bottom edge 14 of the first section 10 and the second top edge 22 of the second section 20. It should be understood that the base member 100 is manufactured as a single unit structure with the first section 10, the second section 20, and the middle section 30.

Further, the wrapping package 1000 includes a flap member 200 extending outwardly from the first section 10 of the base member 100. The flap member 200 includes a first end portion 202 and a second end portion 204 opposite to the first end portion 202 (as shown in FIGS. 1A and 2A). The first end portion 202 is coupled with the first top edge 12 of the first section 10. Specifically, the flap member 200 is permanently sealed with the first top edge 12 of the first section 10 at the first end portion 202. The second end portion 204 is capable of being resealably coupled with the first bottom edge 14 of the first section 10. Specifically, the flap member 200 includes a first sealing element 206 disposed on the second end portion 204 for resealably coupling the second end portion 204 of the flap member 200 with the first bottom edge 14 of the first section 10 for configuring a first enclosure 208 (as shown in FIG. 3) between the flap member 200 and the first section 10 of the base member 100. More specifically, the flap member 200 may be folded along an axis X-X′ (as shown in FIGS. 1A and 2A) for allowing the resealable coupling of the second end portion 204 of the flap member 200 with the first bottom edge 14 of the first section 10 for configuring the first enclosure 208. The first enclosure 208 is capable of storing one or more unused articles, such as a new tampon assembly 2000 including a new tampon 2002 and a new tampon applicator 2004 (as shown in FIG. 2B). However, it will be evident to a person skilled in the art that the first enclosure 208 may store a new tampon such as the new tampon 2002 alone or in combination with a new tampon applicator such as the new tampon applicator 2004.

Further, it will be evident to a person skilled in the art that the first sealing element 206 may be disposed on the second end portion 204 of the flap member 200 by any means known in the art, such as attaching by glue, stitching, and the like. It will also be evident to a person skilled in the art that the first sealing element 206 may either be an adhesive strip such as a peel and stick strip, a two-sided adhesive strip and the like, or a layer of glue.

The wrapping package 1000 further includes a covering member 300 coupled with the second section 20 at three edges of the second plurality of edges of the second section 20 for configuring a second enclosure 302 between the covering member 300 and the second section 20 of the base member 100. Specifically, the covering member 300 is coupled with the second section 20 of the base member 100 at the second pair of side edges and the second bottom edge 24 of the second section 20 for configuring a second enclosure 302 between the covering member 300 and the second section 20 of the base member 100 (as shown in FIG. 2A). More specifically, the covering member 300 is coupled with the second section 20 by permanent sealing at the second pair of side edges and the second bottom edge 24 of the second section 20.

The second enclosure 302 is capable of receiving, storing, and concealing a used tampon 2006 (as shown in FIG. 3). However, it will be evident to a person skilled in the art that the second enclosure 302 may store a used tampon such as the used tampon 2006 alone or in combination with a new tampon applicator removed from a tampon assembly, such as the new tampon assembly 2000, after inserting a new tampon, such as the new tampon 2002, into vagina of a woman. In the present embodiment of the present disclosure, the second enclosure 302 is capable of storing the used tampon 2006, and the new tampon applicator 2004 after being used for inserting the new tampon 2002 into vagina of a woman.

The wrapping package 1000 may include a liner member (not shown) at an inner surface (not shown) of the second enclosure 302 for lining the second enclosure 302 from inside in order to conceal the used tampon 2006, and the new tampon applicator 2004 after being used by a woman. Preferably, the liner member is composed of a leak-proof material, and accordingly, the wrapping package 1000 is capable of preventing leakage of body fluids retained in the used tampon 2006 held within the second enclosure 302. Further, the use of the liner member provides opacity to the second enclosure 302 for concealing the used tampon 2006, and the new tampon applicator 2004 after being used by the woman, in a proper manner.

In the present embodiment of the present disclosure, the covering member 300 also includes a second sealing element 304 disposed on an outer surface 306 (hereinafter referred to as “surface 306”) of the covering member 300 (as shown in FIGS. 1A and 2A). More specifically, the second sealing element 304 may be disposed at a bottom edge (not shown) of the surface 306 of the covering member 300. Suitable examples of the second sealing element 304 may include, but are not limited to, a layer of glue; an adhesive strip such as a peel and stick strip, a two-sided adhesive strip, and the like; and a tab of tape. Further, it will be evident to a person skilled in the art that the second sealing element 304 may be disposed on the surface 306 of the covering member 300 by any means known in the art, such as attaching by glue, stitching, and the like.

As described above, the wrapping package 1000, as shown in FIGS. 1A, 2A, 2B and 3, is in the open configuration that is achieved by rolling the wrapping package 1000 of FIG. 1B. Specifically, FIG. 1B depicts a front view of the wrapping package 1000, storing the new tampon assembly 2000 including the new tampon 2002 and the new tampon applicator 2004, in the closed configuration (i.e., the rolled configuration), in accordance with an embodiment of the present disclosure.

More specifically, the base member 100 is capable of being rolled from the first section 10 towards the second section 20, such that the second sealing element 304 resealably couples with a rear surface 102, and more particularly, a portion (not numbered) of the rear surface 102, of the base member 100 for enabling the wrapping package 1000 to assume the closed configuration (as shown in FIG. 1B). In use, the wrapping package 1000 may be opened by pulling apart the second section 20 and the covering member 300 from the rear surface 102 of the base member 100 for enabling the wrapping package 1000 to assume the open configuration as shown in FIGS. 1A, 2A, 2B and 3). Accordingly, the wrapping package 1000 may be provided in the closed configuration as illustrated in FIG. 1B, to a woman. Subsequently, the woman may then open/ unroll the wrapping package 1000 for a use thereof.

After opening the wrapping package 1000, the new tampon assembly 2000 may be accessed from the first enclosure 208 by decoupling the second end portion 204 of the flap member 200, as explained in conjunction with FIG. 3.

FIG. 3 depicts a front view of the wrapping package 1000 having the used tampon 2006 received in the second enclosure 302. Further, the flap member 200 may be decoupled and lifted upwardly for withdrawing the new tampon assembly 2000 for obtaining the new tampon 2002 therefrom. More specifically, the second end portion 204 of the flap member 200 may be decoupled from the first bottom edge 14 of the first section 10 of the base member 100 by pulling apart the second end portion 204 of the flap member 200 away from the first bottom edge 14 of the first section 10. The new tampon applicator 2004 once used for inserting the new tampon 2002 in vagina of a woman may then be inserted into the second enclosure 302.

The base member 100 of the wrapping package 1000 is capable of being rolled from the second section 20 towards the first section 10. Specifically, the first sealing element 206 resealably couples with the rear surface 102, and more particularly, a portion (not numbered) of the rear surface 102, of the base member 100 for enabling the wrapping package 1000 to assume the closed configuration prior to disposing the wrapping package 1000 (as shown in FIGS. 4A and 4B). However, for the sake of illustration of the present embodiment of the present disclosure, the used tampon 2006 and the new tampon applicator 2004 are not shown in FIGS. 4A and 4B.

Further, the second section 20 of the base member 100 may be folded up to a portion (not numbered) of the middle section 30 of the base member 100 prior to rolling the base member 100 from the second section 20 towards the first section 10. Specifically, the second section 20 may be folded and resealably coupled with the portion of the middle section 30 with the help of the second sealing element 304. More specifically, the second sealing element 304 may be pressed against the portion of the middle section 30 for sealing the second enclosure 302. Accordingly, the second sealing element 304 serves as a first sealing mechanism for effectively concealing the used tampon 2006 and the new tampon applicator 2004 within the second enclosure 302. It should be understood that the second section 20 may be folded tightly up to a point where the used tampon 2006 and the new tampon applicator 2004 are held within the second enclosure 302 depending on respective sizes of the used tampon 2006 and the new tampon applicator 2004, and volume of the body fluids absorbed by the used tampon 2006.

Furthermore, the second section 20 may further be rolled in an upward direction towards the flap member 200, as shown by an arrow in FIG. 4A. However, it may be evident to a person skilled in the art that the wrapping package 1000 may not be required to be completely rolled, if the liner member possesses sufficient opaqueness.

FIG. 4B depicts a perspective view of the wrapping package 1000 in the closed configuration, i.e., a completely rolled configuration. More specifically, the wrapping package 1000 may assume such closed configuration when the second section 20 completely rolls towards the flap member 200, and the second end portion 204 of the flap member 200 resealably couples with a portion (not numbered) of the rear surface 102 of the base member 100 with the help of the first sealing element 206. More specifically, the first sealing element 206 may be pressed against the portion of the rear surface 102 for sealing the entire wrapping package 1000. Accordingly, the first sealing element 206 serves as a second sealing mechanism for effectively concealing the used tampon 2006 and the new tampon applicator 2004, and enfolding the wrapping package 1000. The wrapping package 1000 may thereafter be disposed in a convenient manner. In case the second sealing element 304 is an adhesive strip, such as a peel and stick strip, the complete rolling of the wrapping package 1000 may not be required, and a tab of tape in place of the second sealing mechanism may be used to seal the wrapping package 1000.

The wrapping package 1000 of the present disclosure is illustrated as a rectangular wrapping package for describing various embodiments of the disclosure. However, it will be evident to a person skilled in the art that the wrapping package 1000 may be configured as a square package, a hexagonal package, and the like. The wrapping package 1000 of the present disclosure may have a height of about three inches and a length of about five inches. However, it will be evident to a person skilled in the art that the dimensions of the wrapping package 1000 may vary depending upon a manufacturer's preference and a user's requirement.

Further, the second enclosure 302 may have a suitable size corresponding to the sizes of the used tampon 2006 and the new tampon applicator 2004, and depending on the volume of the body fluids absorbed by the used tampon 2006. Further, size of the second enclosure 302 may also depend on weight of materials used for manufacturing the wrapping package 1000 and various components thereof. Furthermore, the second enclosure 302 may be provided with a specific width for inserting the one or more used articles there within by simply holding the second section 20 by the second pair of side edges thereof for opening the second enclosure 302. Alternately, a depth may be provided to the second enclosure 302 for opening the second enclosure 302 in case the wrapping package 1000 is heavy.

Furthermore, the wrapping package 1000 and components thereof are composed of a plastic material, such as polyethylene, polypropylene, and the like. Specifically, the base member 100, the flap member 200, and the covering member 300 are composed of a plastic material. More specifically, the base member 100, the flap member 200, and the covering member 300 may be composed as sheets of plastic material. Moreover, the plastic material may be a colored plastic material to identify the absorbency level of the new tampon, for example. In another embodiment of the present disclosure, the wrapping package 1000 and the components thereof may be composed of a biodegradable material, such as a compostable plastic that conforms to the ASTM D6400 standard.

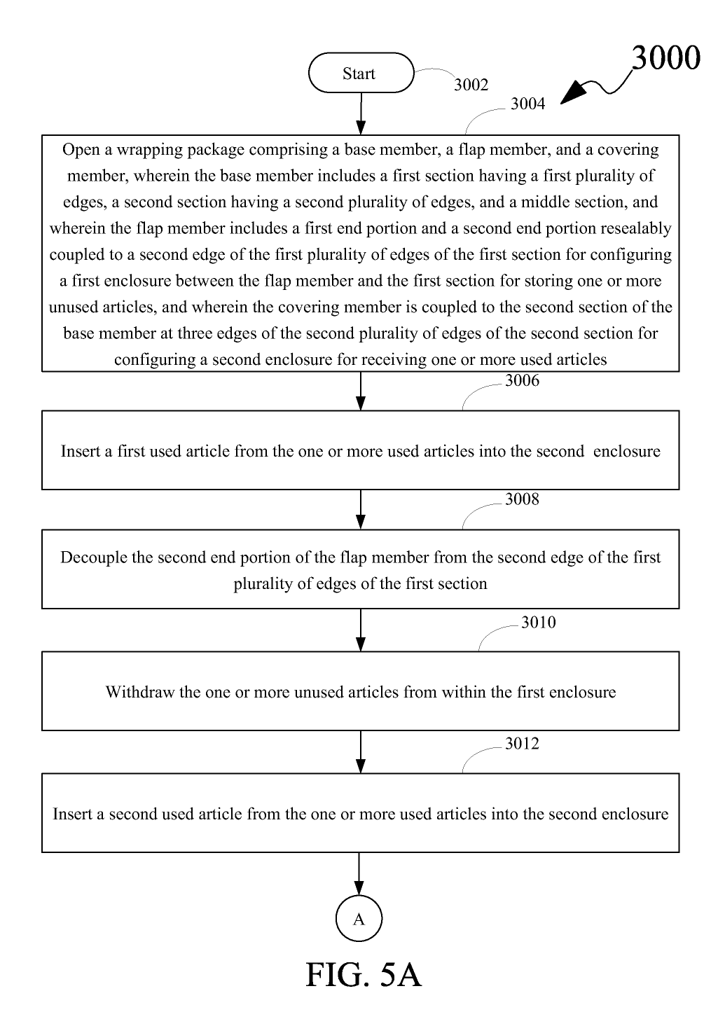

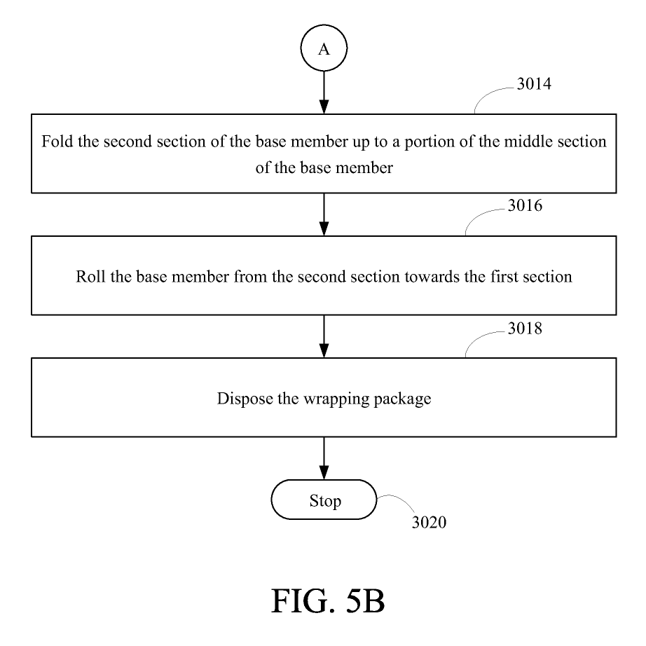

In another aspect of the present disclosure, a method of accessing one or more unused articles and disposing one or more used articles is provided. The method of accessing the one or more unused articles and disposing the one or more used articles is described in conjunction with FIGS. 5A and 5B. Further, reference will be made to wrapping package 1000 of FIGS. 1A, 1B, 2A, 2B 3, 4A, and 4B for describing the method of the present disclosure.

FIGS. 5A and 5B depict a flow diagram of a method 3000 for accessing one or more unused articles such as the new tampon 2002 and the new tampon applicator 2004, and disposing one or more used articles such as the used tampon 2006, in a wrapping package, such as the wrapping package 1000, provided in a closed configuration (i.e., a rolled configuration), according to an embodiment of the present disclosure. The wrapping package 1000 includes a base member, such as the base member 100; a flap member, such as the flap member 200; and a covering member, such as the covering member 300. The base member 100 includes a first section, such as the first section 10, having a first plurality of edges; a second section, such as the second section 20, having a second plurality of edges; and a middle section, such as the middle section 30 configured in between the first section 10 and the second section 20 (as shown in FIGS. 1A, 2A, 2B, and 3).

The flap member 200 extends outwardly from the first section 10 of the base member 100. The flap member 200 includes a first end portion, such as the first end portion 202 and a second end portion, such as the second end portion 204, opposite to the first end portion 202. The first end portion 202 is coupled with a first edge, such as the first top edge 12 of the first plurality of edges of the first section 10. The second end portion 204 is capable of being resealably coupled with a second edge, such as the first bottom edge 14, opposite to the first top edge 12 of the first plurality of edges, for configuring a first enclosure, such as the first enclosure 208, between the flap member 200 and the first section 10 of the base member 100 for storing the new tampon assembly 2000. Specifically, the flap member 200 may be folded along an axis X-X′ for enabling the resealable coupling of the second end portion 204 with the first bottom edge 14 of the first plurality of edges. The flap member 200 further includes a first sealing element, such as the first sealing element 206, disposed on the second end portion 204 of the flap member 200 for resealably coupling the second end portion 204 of the flap member 200 with the first bottom edge 14 of the first plurality of edges (as shown in FIGS. 1A, and 3).

The covering member 300 is coupled with the second section 20 of the base member 100 at three edges of the second plurality of edges of the second section 20 for configuring a second enclosure, such as the second enclosure 302, between the covering member 300 and the second section 20 of the base member 100. The second enclosure 302 is capable of receiving the one or more used articles, such as the used tampon 2006. Further, the covering member 300 includes a second sealing element, such as the second sealing element 304 disposed on an outer surface, such as the surface 306 of the covering member 300 (as shown in FIGS. 1A, 2A, 2B, and 3).

The closed configuration of the wrapping package 1000, prior to use, is achieved when the base member 100 is rolled from the first section 10 towards the second section 20, such that the second sealing element 304 resealably couples with a rear surface, such as the rear surface 102, of the base member 100.

It should be understood that the wrapping package 1000 is explained in detail with reference to FIGS. 1A, 1B, 2, 3, and 4, and accordingly, a description thereof is avoided herein for the sake of brevity.

The method starts at 3002. At 3004, the wrapping package 1000 (provided in the closed configuration) of FIG. 1B is opened, for a use thereof. The wrapping package 1000 is opened by decoupling the second sealing element 304 and pulling apart the second section 20 and the covering member 300 from the rear surface 102 of the base member 100 for enabling the wrapping package 1000 to assume an open configuration of FIG. 2B. Further, a first used article from the one or more used articles, such as the used tampon 2006, may be inserted into the second enclosure 302 of the wrapping package 1000, at 3006 (as shown in FIG. 3).

Subsequently, the second end portion 204 of the flap member 200 is decoupled from the first bottom edge 14 of the first plurality of edges of the first section 10 by pulling apart the second end portion 204 of the flap member 200 away from the first bottom edge 14 of the first plurality of edges of the first section 10, at 3008 (as shown in FIG. 3). Thereafter, the flap member 200 may be lifted and fully extended in order to access the new tampon assembly 2000 from within the first enclosure 208. At 3010, the new tampon assembly 2000 may be withdrawn from within the first enclosure 208. Subsequently, a second used article from the one or more used articles, such as the new tampon applicator 2004, may also be inserted into the second enclosure 302, at 3012. Thereafter, the second section 20 of the base member 100 may be folded up to a portion of the middle section 30 of the base member 100, at 3014. At 3016, the base member 100 may be rolled from the second section 20 towards the first section 10, such that the first sealing element 206 resealably couples with the rear surface 102 of the base member 100. The wrapping package 1000 may then be disposed off at 3018. The method stops at 3020.

The present disclosure provides a wrapping package, such as the wrapping package 1000, which is a compact unit that serves as a dual-purpose tool that allows for storing one or more unused articles, such as a new tampon, and for disposing one or more used articles, such as a used tampon. Specifically, the wrapping package includes separate enclosures for storing the one or more unused articles that need to be used, and the one or more used articles that need to be disposed. Further, the wrapping package is capable of preventing leakage of fluids, such as body fluids, retained in a used article such as a used tampon. Accordingly, the use of the wrapping package provides a hygienic, effective, and convenient way of disposing the one or more used articles. Further, sealing mechanisms of the wrapping package aid in wrapping the one or more used articles in an effective manner, such that the one or more used articles are properly concealed and are not visible from outside.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, and to thereby enable others skilled in the art to best utilize the present disclosure and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the claims of the present disclosure.

What is claimed is:

1. A wrapping package comprising:
a base member comprising,
a first section having a first plurality of edges;
a second section having a second plurality of edges, and
a middle section configured in between the first section and the second section;
a flap member extending outwardly from the first section of the base member, the flap member having a first end portion and a second end portion opposite to the first end portion, the first end portion coupled with a first edge of the first plurality of edges of the first section, the second end portion capable of being resealably coupled with a second edge opposite to the first edge of the first plurality of edges for configuring a first enclosure between the flap member and the first section, wherein the first enclosure is capable of storing one or more unused articles; and
a covering member coupled with the second section of the base member at three edges of the second plurality of edges of the second section for configuring a second enclosure between the covering member and the second section, wherein the second enclosure is capable of receiving one or more used articles.

2. The wrapping package of claim 1, wherein the base member is composed of a plastic material.

3. The wrapping package of claim 2, wherein the base member is a sheet of a plastic material.

4. The wrapping package of claim 1, wherein the flap member comprises a first sealing element disposed on the second end portion of the flap member for resealably coupling the second end portion of the flap member with the second edge of the first plurality of edges of the first section.

5. The wrapping package of claim 4, wherein the base member is capable of being rolled from the second section towards the first section, such that the first sealing element resealably couples with a rear surface of the base member for enabling the wrapping package to assume a closed configuration.

6. The wrapping package of claim 1, wherein the covering member comprises a second sealing element disposed on a surface of the covering member.

7. The wrapping package of claim 6, wherein the base member is capable of being rolled from the first section towards the second section, such that the second sealing element resealably couples with a rear surface of the base member for enabling the wrapping package to assume a closed configuration.

8. The wrapping package of claim 1, wherein the covering member and the flap member are composed of a plastic material.

9. The wrapping package of claim 1, wherein the one or more unused articles is a tampon.

10. The wrapping package of claim 1, wherein the one or more unused articles is a tampon assembly comprising a tampon and a tampon applicator.

11. The wrapping package of claim 1, wherein the one or more used articles is a tampon.

12. The wrapping package of claim 1, wherein the one or more used articles is a tampon applicator.

13. A method of accessing one or more unused articles and disposing one or more used articles, the method comprising:
opening a wrapping package provided in a closed configuration, the wrapping package comprising,
a base member comprising,
a first section having a first plurality of edges,
a second section having a second plurality of edges, and
a middle section configured in between the first section and the second section,
a flap member extending outwardly from the first section of the base member, the flap member having a first end portion and a second end portion opposite to the first end portion, the first end portion coupled with a first edge of the first plurality of edges of the first section, the second end portion resealably coupled with a second edge opposite to the first edge of the first plurality of edges for configuring a first enclosure between the flap member and the first section for storing the one or more unused articles, the flap member comprising a first sealing element disposed on the second end portion of the flap member for resealably coupling the second end portion of the flap member with the second edge of the first plurality of edges, and
a covering member coupled with the second section of the base member at three edges of the second plurality of edges of the second section for configuring a second enclosure between the covering member and the second section, the second enclosure capable of receiving the one or more used articles, the covering member comprising a second sealing element disposed on a surface of the covering member,
wherein the wrapping package assumes the closed configuration when the base member is rolled from the first section towards the second section and the second sealing element resealably couples with a rear surface of the base member, and wherein the wrapping package is opened by decoupling the second sealing element and pulling apart the second section and the covering member from the rear surface of the base member for enabling the wrapping package to assume an open configuration;
inserting a first used article from the one or more used articles into the second enclosure;
decoupling the second end portion of the flap member from the second edge of the first plurality of edges by pulling apart the second end portion of the flap member away from the second edge of the first plurality of edges;
withdrawing the one or more unused articles from within the first enclosure;
rolling the base member from the second section towards the first section such that the first sealing element resealably couples with the rear surface of the base member; and
disposing the wrapping package.

14. The method of claim 13, further comprising inserting a second used article from the one or more used articles into the second enclosure prior to rolling the base member from the second section towards the first section.

15. The method of claim 14, wherein the second used article is a tampon applicator.

16. The method of claim 13, further comprising folding the second section up to a portion of the middle section of the base member prior to rolling the base member from the second section towards the first section.

17. The method of claim 13, wherein the one or more unused articles is a tampon.

18. The method of claim 13, wherein the one or more unused articles is a tampon assembly comprising a tampon and a tampon applicator.

19. The method of claim 13, wherein the first used article is a tampon.

20. The method of claim 13, wherein the base member is composed of a plastic material.
